(19) United States
(12) Patent Application Publication
(10) Pub. No.: US 2009/0117500 A1
(43) Pub. Date: May 7, 2009

(54) PHOTORESIST STRIP WITH OZONATED ACETIC ACID SOLUTION

(76) Inventors: Roman Gouk, San Jose, CA (US); Steven Verhaverbeke, San Francisco, CA (US)

Correspondence Address: APPLIED MATERIALS/BSTZ;BLAKELY SOKOLOFF TAYLOR & ZAFMAN LLP, 1279 OAKMEAD PARKWAY, SUNNYVALE, CA, 94085-4040, US

(21) Appl. No.: 11/933,661

(22) Filed: Nov. 1, 2007

(51) Int. Cl. G03C 1/805; A61L 2/02; G03C 11/12

(52) U.S. Cl. 430/432; 422/292; 430/463

(57) ABSTRACT

A solution, apparatus, and method for stripping photoresist from a workpiece are disclosed. Embodiments of the invention describe a solution comprising diluted liquid acetic acid and dissolved gaseous ozone. In an embodiment an ozonated liquid acetic acid solution is prepared by dissolving ozone in liquid DI water and then mixing with liquid acetic acid. In another embodiment an ozonated liquid acetic acid solution is prepared by mixing liquid DI water and liquid acetic acid and then dissolving ozone. The ozonated liquid acetic acid solution is used to strip a layer of photoresist from a workpiece with improved performance.

### BACKGROUND OF THE INVENTION

1. Field of the Invention

The present invention relates to the field of semiconductor processing and manufacturing. More particularly this invention relates to the area of stripping photoresist from a workpiece.

2. Discussion of Related Art

As semiconductor devices become increasingly more complex and the technology nodes continue to shrink to 65 nm and below, photomask manufacturing is also becoming more critical and requires new approaches and techniques. As shown in FIG. 1A conventional photomask manufacturing typically begins with a transparent substrate 102, such as quartz. A phase shift layer 104 such as MoSiₓ is disposed over the quartz substrate 102. A Cr layer 106 is disposed over phase shift layer 104, and an antireflective (ARC) coating 108, such as CrOₓ, is disposed over the Cr layer 106. Finally, a photoresist layer 110 is formed over ARC coating 108.

As shown in FIG. 1B, photoresist layer 110 is exposed with an electron (or laser) beam and developed to form a predetermined circuitry pattern in the photoresist layer 110. Thereafter, as shown in FIG. 1C, selective etch chemistries are utilized to selectively etch the ARC layer 108, Cr layer 106, and the phase shift layer 104 while using the photoresist pattern 110 as an etching mask. The remaining first electron beam photoresist layer 110 is then stripped in FIG. 1D.

Then a second photoresist layer 112 is formed on the patterned ARC layer 108 and quartz substrate 102, as shown in FIG. 1E. Photoresist layer 112 is exposed with an electron (or laser) beam and developed to form a second predetermined circuitry pattern as shown in FIG. 1F. Thereafter, the exposed portions of ARC layer 108 and Cr layer 106 are removed by using the second photoresist pattern 112 as the etching mask, as shown in FIG. 1G. Finally, the remaining photoresist 112 is stripped in FIG. 1H.

In a conventional photoresist wet-stripping method, a series of operations can be performed to remove the photoresist. In some methods, a stripping solution can be applied to the photomask, followed by a cleaning solution, followed by a rinsing solution, followed by drying of the photomask. The common and state-of-the-art photoresist stripping solution is the sulfuric acid (H₂SO₄) and hydrogen peroxide (H₂O₂) mixture (known as the SPM) strip. The cleaning solution, can be, for example, DI water or an ammonium hydroxide (NH₄OH) and hydrogen peroxide mixture (APM). Examples of APMs include Standard Clean-1 (SC-1) and AM-clean™ (available from Applied Materials, Inc., Santa Clara, Calif.) which is a solution resulting from the mixture of ammonium hydroxide (NH₄OH), hydrogen peroxide (H₂O₂), water (H₂O), a chelating agent, and a surfactant. The mixture of ammonium hydroxide, surfactant and chelating agent is sold in a proprietary blend known as AM1 (available from Mitsubishi Chemical Corporation, Tokyo, Japan). The rinsing solution can typically be, for example, DI water. Drying can be performed by spin drying and the like techniques.

The SPM stripping solution offers a high photoresist strip rate (>1 μm/min) and high conductivity (>200 mS/cm) at a pH of 0 to 1. However, the SPM stripping solution is problematic in that it leaves sulfur residues, which contributes to haze formation under high intensity UV exposure, which in turn requires photomask re-cleaning and causes yield losses. Therefore, the photomask industry is in the need of a sulfur-free stripping solution and process.

One state-of-the art sulfur-free photoresist strip utilizes an ozonated DI water stripping solution. While this method is sulfur-free, it poses a number of additional issues. Pure DI water has a maximum gaseous ozone (O₃) solubility limit of approximately 50 ppm at room temperature. Ozone is the primary stripping component in such a solution. A typical ozonated DI water stripping solution containing approximately 20 ppm dissolved ozone has a photoresist strip rate of approximately 7.5 nm/min, which is much lower than that of the SPM photoresist strip rate.

Another issue is that an ozonated DI water stripping solution has a much lower conductivity than the SPM strip and therefore can cause ESD damage as a result of the build up of static electricity in solution. Ozonated DI water has a conductivity less than 10 μS/cm. CO₂ gas can be dissolved into the ozonated DI water solution in order to increase the conductivity up to an amount of approximately 40 μS/cm. While the addition of CO₂ gas helps alleviate the issue of ESD damage, CO₂ gas displaces the ozone gas in solution and additionally lowers the already low strip rate of the ozonated DI water strip. Thus, the photomask industry is in the need of a sulfur-free stripping process and solution which has an acceptable strip rate and high conductivity so that ESD damage is minimized.

In addition, ozonated DI water is a high surface tension liquid (72 dyne/cm). This causes poor surface wetting especially for hydrophobic surfaces such as post-etch photoresist on masks. Poor wetting also prevents the liquid from wetting contact holes and also creates problems with photoresist removal on vertical edges of photomasks. Most photomasks are square and good wetting is needed to cover the edges with the liquid when used in a spin-on type of application (which is typical for mask strip/clean tools). Thus, the photomask industry is in the need of a sulfur-free stripping process and solution which has an acceptable surface tension.

Thus, what is needed is a sulfur-free photoresist strip process with improved performance.

### SUMMARY OF THE INVENTION

Embodiments of the present invention disclose a solution, apparatus, and method for stripping photoresist from a workpiece. Embodiments of the invention describe a solution comprising liquid acetic acid and the associated benefits of the compound. The solution consists of the solution resulting from dissolving gaseous ozone in liquid acetic acid. The solution may further comprise liquid DI water. In an embodiment liquid DI water is ozonated and then mixed with liquid acetic acid. In another embodiment liquid DI water and liquid acetic acid are mixed and then ozonated. In an embodiment of the invention, a layer of photoresist is stripped from a workpiece using an ozonated liquid acetic acid solution. In an embodiment, the workpiece is then rinsed with a liquid acetic acid solution, followed by an APM clean, and rinsed with DI water with dissolved CO₂ gas.

### BRIEF DESCRIPTION OF THE DRAWINGS

FIG. 1A-1H are side view illustrations of a conventional photomask manufacturing method.

FIG. 2 is an illustration of a method for processing a photomask.

FIG. 3 is a table illustrating surface tension, ozone concentration, and conductivity based volume ratio of acetic acid in solution.

FIG. 4 is an illustration of the relationship of solution etch rate of a positive photoresist to the volume ratio of acetic acid in solution.

FIG. 5 is a schematic illustration of an apparatus for supplying an ozonated liquid acetic acid solution to a photomask.

FIG. 6 is a schematic illustration of an apparatus for supplying an ozonated liquid acetic acid solution to a photomask.

FIG. 7 is an illustration of a method for supplying an ozonated liquid acetic acid solution to a photomask utilizing the apparatus of FIG. 5.

FIG. 8 is an illustration of a method for supplying an ozonated liquid acetic acid solution to a photomask utilizing the apparatus of FIG. 6.

FIG. 9 is an illustration of a method for processing a photomask.

FIG. 10 is an illustration of a method for processing a photomask.

### DETAILED DESCRIPTION OF THE PRESENT INVENTION

Embodiments of the present invention disclose a solution, apparatus, and method for stripping photoresist from a workpiece.

Various embodiments described herein are described with reference to figures. However, certain embodiments may be practiced without one or more of these specific details, or in combination with other known methods and configurations. In the following description, numerous specific details are set forth, such as specific configurations, compositions, and processes, etc., in order to provide a thorough understanding of the present invention. In other instances, well-known semiconductor processes and manufacturing techniques have not been described in particular detail in order to not unnecessarily obscure the present invention. Reference throughout this specification to “one embodiment” or “an embodiment” means that a particular feature, configuration, composition, or characteristic described in connection with the embodiment is included in at least one embodiment of the invention. Thus, the appearances of the phrase “in one embodiment” or “an embodiment” in various places throughout this specification are not necessarily referring to the same embodiment of the invention. Furthermore, the particular features, configurations, compositions, or characteristics may be combined in any suitable manner in one or more embodiments.

In an embodiment, an ozonated liquid acetic acid stripping solution of the present invention consists of the solution resulting from liquid acetic acid (CH₃COOH), liquid DI water (H₂O), and gaseous ozone (O₃) dissolved therein. The stripping solution offers several advantages over the ozonated DI water stripping solution including higher conductivity resulting in less ESD damage, lower surface tension resulting in better surface wetting, and higher solubility of ozone gas which results in a higher strip rate of photoresist. Additionally, because the solubility of ozone gas in liquid acetic acid is higher than in liquid DI water, the addition of a scavenging agent such as CO₂ gas may not be required to prevent the rapid decomposition of dissolved ozone in solution. For example, in a conventional ozonated DI water stripping solution approximately 10% of the dissolved ozone gas decomposes after one minute in solution, and approximately 90% of the dissolved ozone gas is decomposed after eight minutes in solution. The addition of acetic acid according to embodiments of the present invention aids to prevent decomposition of ozone in solution. Embodiments of the liquid stripping method of the present invention also offer the additional benefit over vapor stripping methods in that the liquid stripping method inherently controls ozone gas concentration in the solution through solubility limits, and plant operation with liquid stripping methods is less hazardous than operating with vapor stripping methods.

In one embodiment, the ozonated liquid acetic acid solution is diluted with DI water, and the solution comprises a liquid acetic acid volume percent of approximately 0.1% to 50%. In an embodiment, the ozonated liquid acetic acid solution comprises approximately 0.1% to 25% liquid acetic acid volume percent. In such an embodiment, when the liquid acetic acid and DI water are mixed and then ozonated, the solution has a concentration up to approximately 87 ppm dissolved ozone. Alternatively, when the DI water is ozonated prior to mixing with the liquid acetic acid, the solution has a concentration up to approximately 50 ppm ozone.

In another embodiment, the ozonated liquid acetic acid solution is diluted with DI water, and the solution comprises a liquid acetic acid volume percent above 50% and less than 70%. In an embodiment, the ozonated liquid acetic acid solution comprises above 60% and less than 70% liquid acetic acid volume percent. In such an embodiment, when the liquid acetic acid and DI water are mixed and then ozonated, the solution has a dissolved ozone concentration greater than 140 ppm and less than 155 ppm.

In an embodiment, a rinsing solution of the present invention also consists of liquid acetic acid. Acetic acid is a solvent and helps to remove organics. Additionally, the lower surface tension and higher wetability of hydrophobic surfaces by acetic acid compared to DI water is useful for removing contaminants from the surface of the photomask after a stripping operation. In an embodiment the rinsing solution includes liquid DI water and liquid acetic acid. In another embodiment the rinsing solution includes liquid DI water, liquid acetic acid, and CO₂ gas. The CO₂ gas helps remove residual O₃ radicals form the surface of the photomask after a stripping operation. The mixture of liquid acetic acid and liquid DI water also results in a higher conductivity solution to reduce ESD damage during the rinse operation.

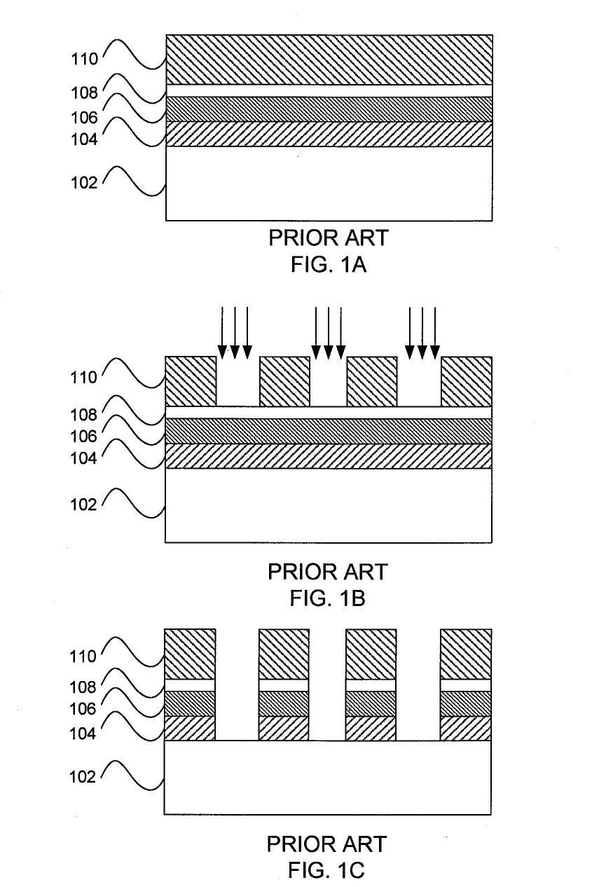

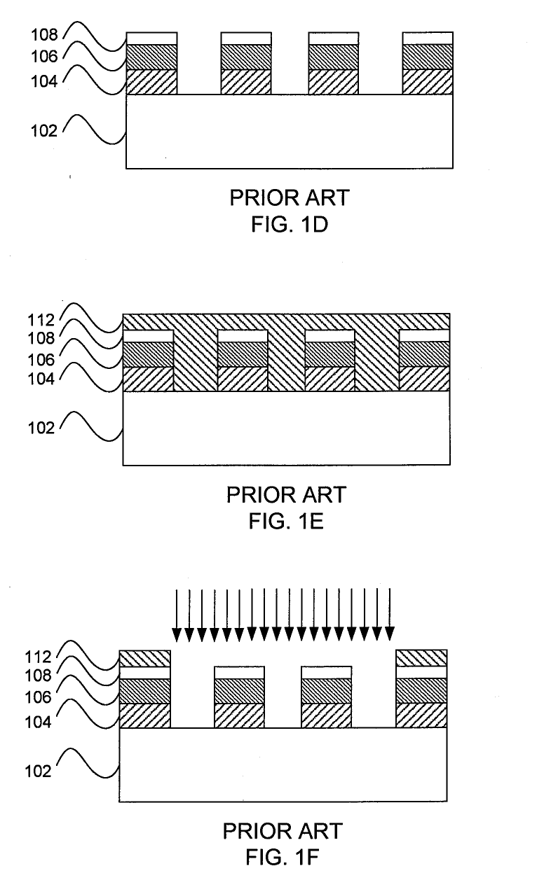

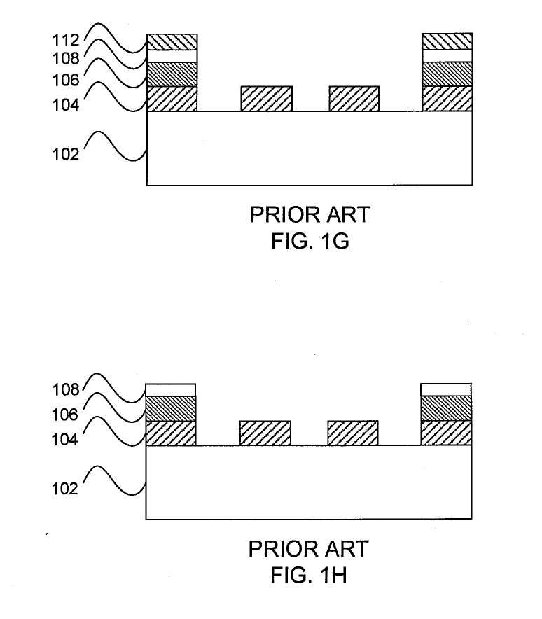

Embodiments the invention described herein are specifically useful for increasing the efficiency of photoresist stripping from a single photomask, but they may also be used in batch operations. The compositions and processes are specifically useful for the removal of photoresist from the top surface of a photomask that is disposed during conventional photomask manufacturing, such as layers 110 and 112 in FIG. 1. It is to be appreciated, that while embodiments of the invention are described with respect to removal of photoresist from a photomask, that embodiments of the invention could also be practiced with removal of organic layers from other workpieces such as silicon or GaAs wafers.

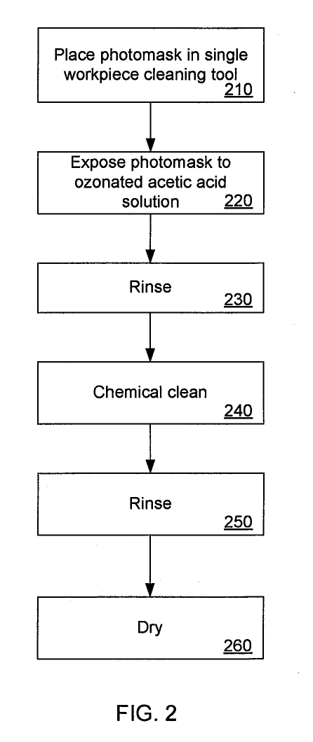

FIG. 2 illustrates an embodiment of the present invention for a method of stripping a layer of photoresist from a photomask. A photomask, such as described in FIG. 1 with a layer of photoresist (110 or 112) to be removed, is placed on a photomask holder in a single workpiece cleaning tool (See FIG. 2, 210). Then as shown in block 220 an ozonated liquid acetic acid stripping solution is dispensed onto the top surface of the photomask for a sufficient amount of time to substantially remove the layer of photoresist.

In one embodiment, the ozonated liquid acetic acid solution is diluted with DI water, and the solution comprises a liquid acetic acid volume percent of approximately 0.1% to 50%. In one embodiment, the ozonated liquid acetic acid solution comprises a liquid acetic acid volume percent of approximately 0.1% to 25%, a surface tension below approximately 72 dyne/cm, conductivity greater than 100 μS/cm, a flash point above approximately 100° C., and a pH less than 4. When the liquid acetic acid and DI water are mixed and then ozonated, the solution has a concentration up to approximately 87 ppm dissolved ozone. When the DI water is ozonated prior to mixing with the liquid acetic acid, the solution has a concentration up to approximately 50 ppm ozone. Such embodiments where the solution comprises a liquid acetic acid volume percent of approximately 0.1% to 50%, are preferable for improving upon the conductivity of a state-of-the-art ozonated DI water stripping solution.

While pure acetic acid is effectively non-conductive, the addition of liquid acetic acid to DI water significantly increases the conductivity of the solution, even at a low percentage of 0.1%. It has been discovered that when the conductivity of the solution is increased above approximately 100 μS/cm the build up of static electricity in the solution and related ESD damage is significantly reduced. In one embodiment, the solution comprises a sufficient amount of liquid acetic acid volume percent to obtain a conductivity greater than approximately 100 μS/cm. In one embodiment, the solution comprises a liquid acetic acid volume percent of approximately 0.1% or more and possesses a conductivity greater than approximately 100 μS/cm.

In another embodiment, where a more aggressive stripping solution is desired, the ozonated liquid acetic acid solution comprises a liquid acetic acid volume percent greater than 50% and less than 70%. In one embodiment, the ozonated liquid acetic acid solution comprises a liquid acetic acid volume percent greater than 60% and less than 70%, an ozone concentration greater than 140 ppm and less than 155 ppm, a surface between approximately 38 dyne/cm and 41 dyne/cm, a conductivity greater than 100 μS/cm, a flash point above approximately 60° C. to 70° C., and a pH less than 4. Such an embodiment may be preferable for removing a layer of photoresist several hundred nm thick in less than five minutes. The backside of the photomask can also be rinsed or stripped at this time by flowing a rinse or stripping solution to the backside of the photomask.

Following the stripping operation, the top surface of the photomask is rinsed in order to remove all the chemicals from the surface of the photomask as set forth in block 230. The backside of the photomask can also be rinsed at this time. In an embodiment, the rinse solution comprises liquid acetic acid. The acetic acid provides low surface tension and high wetability of hydrophobic surfaces, and is additionally useful dissolving and removing residual particles of photoresist. In an embodiment the rinse solution comprises liquid acetic acid and DI water. The mixture of liquid acetic acid and liquid DI water results in a higher conductivity solution to reduce ESD damage during the rinse operation. In another embodiment, the rinse solution is DI water.

Following the rinse operation the top surface of photomask 656 is exposed to a chemical clean as set forth in block 240. In an embodiment the chemical clean is an ammonia/peroxide mixture (APM) chemical clean, which removes any acetic acid residues on the surface of the photomask. In an embodiment the APM concentration is 1:2:50-500 (ammonia or AM1: H₂O₂: DI water). The backside of the photomask can also be rinsed or exposed to the APM chemical clean at this time.

Then the photomask is exposed to a second rinsing operation at block 250. In an embodiment, the rising solution comprises DI water and CO₂ gas. The CO₂ gas helps remove residual O₃ radicals from the surface of the photomask. Following the rinsing operation, the flow of all solutions is stopped, and the photomask is spun dry at very high rotation speeds as set forth in block 260. If desired, N₂ and/or IPA vapor may be blown on the photomask to assist in the drying of the photomask.

Acetic acid, one of the simplest carboxylic acids, is a hydrophilic solvent. Acetic acid can dissolve not only polar compounds, but also non-polar compounds. It readily mixes with many other polar and non-polar solvents such as water. Pure acetic acid has a flash point of approximately 40° C. Thus, in one embodiment liquid acetic acid is diluted with greater than 20 volume percent liquid DI water to increase the flash point of the solution above approximately 60° C. to 70° C., which would be an acceptable minimum range for room temperature operation with the solution in non-explosion-proofed equipment. In another embodiment, liquid acetic acid is diluted with greater than 50 volume percent liquid DI water to increase the flash point of the solution above 100° C. In addition, utilizing embodiments of the invention, the ozonated liquid acetic acid solution can be applied above room temperature or to a heated workpiece without the concern of a fire hazard.

Another advantage of diluting the acetic acid with DI water is that while most currently available mask spinning chambers are coated with a generally chemically resistant fluoropolymer such as PVDF or PPCTFE, it is preferable to not continuously expose the chambers to highly acidic conditions. In addition, there is a concern for protecting other parts not coated with chemically resistant materials. Acetic acid is regarded as a weak acid with a pH of approximately 2.4, and DI water has a neutral pH of approximately 7. As the ratio of acetic acid to DI water increases, the pH of the solution decreases. In one embodiment, the concentration of liquid acetic acid in solution is less than 70% in order to help preserve the integrity of the mask spinning chamber.

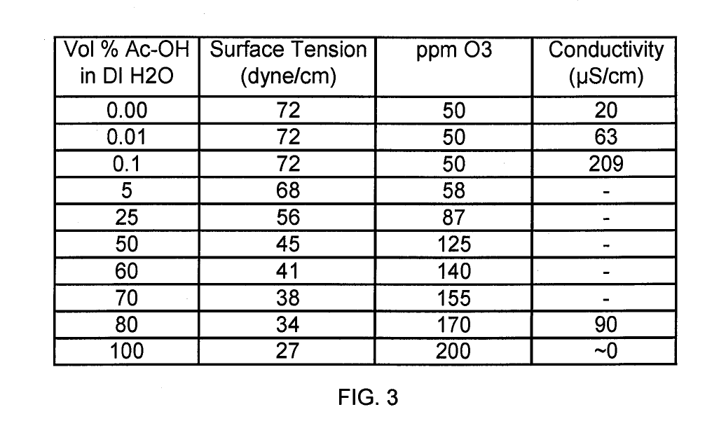

Alternatively, while it is preferable to dilute the liquid acetic acid solution in order to prevent excessive chamber material decomposition as discussed above, it is also preferable to dilute acetic acid to obtain a sufficient conductivity. This is particularly important because static electricity can build up in the stripping or rinsing solution when the photomask is being rotated between 10 rpm to 3000 rpm. It has been discovered that when the conductivity of the stripping or rinsing solution is increased above approximately 100 μS/cm the build up of static electricity in the solution and related ESD damage on the photomask is significantly reduced. FIG. 3 provides conductivity data for a liquid acetic acid solution diluted with DI water. As shown in FIG. 3, the addition of liquid acetic acid to DI water significantly increases the conductivity of the solution, even at a low percentage of 0.1%. FIG. 3 also shows that pure acetic acid is effectively non-conductive, and at high volume percentages of approximately 80% acetic acid and above the conductivity is below 100 μS/cm. In an embodiment, the concentration of liquid acetic acid in solution is approximately 0.1% or above, and less than 70% in order to obtain a conductivity above approximately 100 μS/cm.

In another aspect, acetic acid lowers the surface tension of the stripping and rinsing solution. Pure acetic acid has a surface tension of approximately 27.4 dyne/cm, whereas pure DI water has a surface tension of approximately 72.0 dyne/cm. Surface tension bears an approximate linear relationship to volume percent acetic acid in solution. Thus, as acetic acid concentration in the solution increases, surface tension linearly decreases. The lower surface tension of acetic acid provides good surface wetting which is needed for both post etch photoresist stripping and for rinsing chemicals away from the surface post strip. The lower surface tension additionally helps strip photoresist on photomask edges and in contact holes.

In another aspect, acetic acid increases the solubility of ozone in the liquid stripping solution. Pure acetic acid has a gaseous ozone solubility limit of approximately 200 ppm at room temperature, whereas pure DI water has a gaseous ozone solubility limit of approximately 50 ppm at room temperature. Similarly, ozone solubility also bears an approximate linear relationship to volume percent acetic acid in solution. As the acetic acid concentration in the solution increases, ozone solubility linearly increases. One specific advantage of the increased solubility of ozone in acetic acid is that ozone is the primary ingredient responsible for stripping photoresist.

FIG. 3 also provides extrapolated data for surface tension and maximum concentration of dissolved ozone in a liquid water and liquid acetic acid solution. As shown in FIG. 3, in an embodiment, where the stripping solution has a 50 to 100 volume percentage of acetic acid, the stripping solution has a surface tension of approximately 45 dyne/cm to 27 dyne/cm and a maximum dissolved ozone concentration of approximately 125 ppm to 200 ppm. In an embodiment, where the stripping solution has a volume percentage of acetic acid greater than 60% and less than 70%, the stripping solution has a surface tension greater than 38 dyne/cm and less than 41 dyne/cm, and a maximum dissolved ozone concentration of greater than 140 ppm and less that 155 ppm.

In an embodiment, where the stripping solution has a 0.1 to 50 volume percentage of acetic acid, the stripping solution has a surface tension of approximately 72 dyne/cm to 45 dyne/cm and a maximum dissolved ozone concentration of approximately 50 ppm to 125 ppm. However, it is to be appreciated that the dissolved ozone concentration can be less than the maximum solubility amounts. This is particularly true if ozone is dissolved into either the liquid DI water or liquid acetic acid prior to mixing the two liquids. In an embodiment, the stripping solution has 50% volume percentage acetic acid or less and less than 100 ppm ozone. In an embodiment, where the stripping solution has a 0.1 to 25 volume percentage of acetic acid, the stripping solution has a surface tension of approximately 61 dyne/cm to 72 dyne/cm, and a maximum ozone concentration of approximately 87 ppm when the liquid acetic acid and DI water are mixed prior to ozonating, but a maximum ozone concentration less than approximately 50 ppm when the DI water is ozonated prior to mixing with the liquid acetic acid. Alternatively, the liquid acetic acid can be ozonated prior to mixing with the DI water.

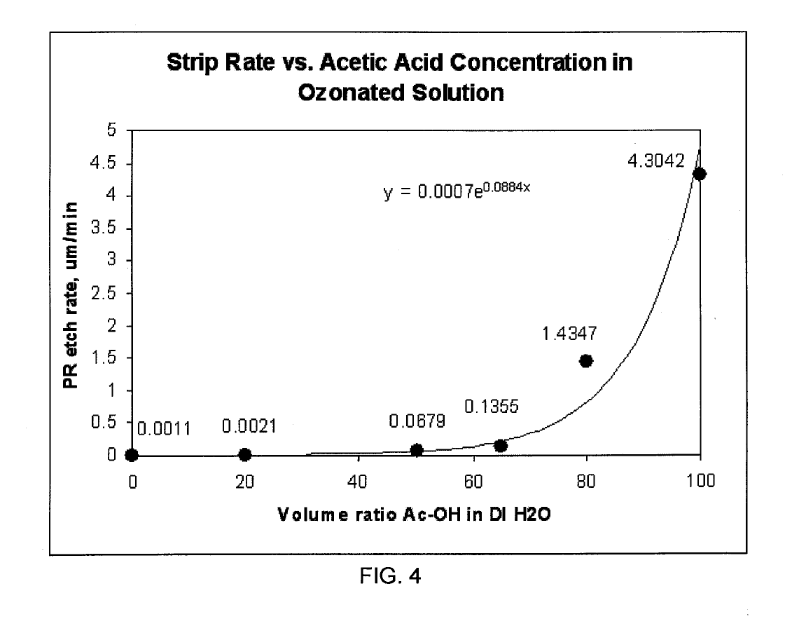

FIG. 4 illustrates the relationship of solution strip rate of a positive photoresist to the concentration of liquid acetic acid in a liquid water and acetic acid solution, where ozone concentration is at the maximum solubility limits as provided in FIG. 3. In one embodiment, where the stripping solution is 100 percent liquid acetic acid having approximately 200 ppm ozone dissolved, a photoresist strip rate of approximately 4.3 μm/min is achieved. This is a substantial increase compared to a 100 percent liquid DI water stripping solution having approximately 50 ppm ozone dissolved, where the photoresist strip rate is approximately 0.0011 μm/min. As shown in FIG. 4, at approximately 80% acetic acid, the stripping solution demonstrates a photoresist strip rate greater than 1 μm/min (i.e. less than 1 minute to strip a 1 μm thick layer of photoresist). At approximately 60% acetic acid, the stripping solution demonstrates a photoresist strip rate of approximately 0.1 μm/min (i.e. approximately 10 minutes to strip a 1 μm thick layer of photoresist).

It has been discovered that at 70% volume acetic acid and above, there is no practical benefit to the increased amount of acetic acid in the solution. In one aspect, a deposited photoresist layer (such as layer 110 or 112 of FIG. 1) is rarely greater than 1 μm thick in conventional photomask processing technology, and therefore there is no practical benefit to having a photoresist strip rate greater than 1 μm/min. In another aspect, an etch process that lasts less than one minute may not provide adequate homogeneity in either a spin-on or batch stripping process. Accordingly, in one embodiment it is preferable to strip a layer of photoresist in less than five minutes, but greater than one minute. In one embodiment, a 300 nm thick layer of photoresist (such as layer 110 or 112 of FIG. 1) is removed from a photomask in greater than one minute and less than five minutes utilizing an ozonated liquid acetic acid stripping solution comprising greater than 50% and less than 70% acetic acid.

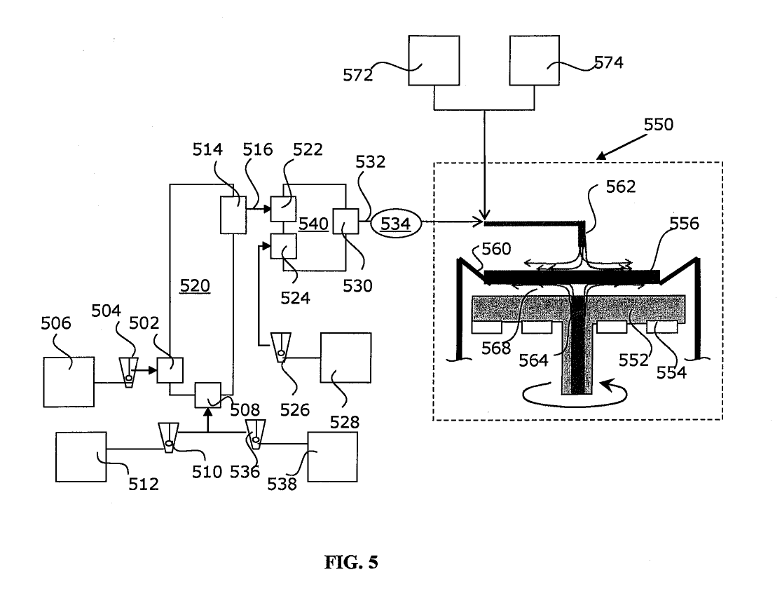

FIG. 5 is an illustration of one embodiment of an apparatus for supplying an ozonated liquid acetic acid solution to a photomask. As shown in FIG. 5 liquid acetic acid is added after the liquid DI water is ozonated. Such an embodiment may be preferable for preparing a diluted ozonated liquid acetic acid solution having a higher conductivity than is achievable with state-of-the-art ozonated DI water solutions. In addition, the apparatus is compatible with existing configurations for preparing ozonated DI water solutions.

An ozonator 520 includes an input port 502, connected to a flow valve 504, which is flow connected to a liquid DI water source 506. In an embodiment, liquid DI water source 506 provides pure liquid DI water. In another embodiment, liquid DI water source provides carbonated liquid DI water. Ozonator 520, further includes input port 508, connected to a flow valve 510, which is flow connected to an ozone gas source 512. In an embodiment, ozonator 520 further includes a flow valve 536 connected to input port 508, and further flow connected to an additional gas source 538, such as a CO₂ gas. Ozonator 520 can be a commercially available unit that enables a gas to be dissolved into a liquid. For example ozonator 520 can be a bubbler system, a venture device that enables a gas to be dissolved into a liquid flow at a gas pressure less than the pressure of the liquid flowing through the conduit, or alternatively a contactor device where gas is fed into a membrane conduit and dissolved into a liquid passing by the membrane. Ozonator 520 further includes an exit port 514 connected to an exit supply line 516, for flowing an ozonated DI water solution.

Exit supply line 516 is also connected to input port 522 of mixer 540. Mixer 540 can be a commercially available unit, such as those including spiraling vortices for the intimate mixing of two liquids. Mixer 540 includes an input port 524 connected to a flow valve 526, which is flow connected to a liquid acetic acid source 528. Mixer 540 further includes an exit port 530 connected to an exit supply line 532, for flowing an ozonated liquid acetic acid solution to a mask spin chamber 550. An ozone ppm monitor 534 can also be included along the supply line 532 in order to monitor ozone concentration of the solution.

Mask spin chamber 550 includes a plate 552 with a plurality of acoustic or sonic transducers 554 located thereon. Plate 552 is preferably made of aluminum but can be formed of other materials such as but not limited to stainless steel and sapphire. The plate is preferably coated with a corrosion resistant fluoropolymer such as PCTFE or PVDF. The transducers 554 are attached to the bottom surface of plate 552 by an epoxy. In an embodiment of the present invention the transducers 554 cover substantially the entire bottom surface of plate 552. The transducers 554 preferably generate megasonic waves in the frequency range above 350 kHz. The specific frequency is dependent on the thickness of the photomask and is chosen by its ability to effectively provide megasonics to both sides of the photomask. But there may be circumstances where other frequencies that do not do this may be ideal for particle removal. In an embodiment, the transducers 554 are piezoelectric devices. The transducers 554 create acoustic or sonic waves in a direction perpendicular to the surface of photomask 556.

A photomask 556 is horizontally held by a photomask support 560 parallel to and spaced apart from the top surface of plate 552. In an embodiment, photomask 556 is held about 3 mm above the surface of plate 552 during cleaning. In an embodiment the photomask 556 is supported on elastomeric pads on posts of support 560 and held in place by gravity. The support 560 can horizontally rotate or spin photomask 556 about its central axis at a rate of between 0 rpm to 3000 rpm. Additionally, in apparatus 550 photomask 556 is placed face up wherein the side of the photomask containing a layer of photoresist (such as layer 110 or 112 of FIG. 1) faces towards a nozzle 562 for spraying a stripping solution, such as ozonated acetic acid, thereon and the backside of photomask 556 faces plate 552.

In an embodiment ozonated acetic acid solution is also feed through a feed port through channel 564 of plate 552 and fills the gap between the backside of photomask 556 and plate 552 to provide a solution filled gap 568 through which acoustic waves generated by transducers 554 can travel to photomask 556. The backside of the photomask 556 can alternatively be rinsed with other solutions during the step. In an embodiment, DI water is fed between the photomask 556 and plate 552. In another embodiment, degassed DI water is fed between the photomask 556 and plate 552 so that cavitation is reduced where acoustic waves are strongest thereby reducing potential damage to photomask 556. While not shown in order to not obscure the present invention it is understood that channel 564 can also be flow connected to the supply line 532, and additionally nozzle 562 and channel 564 can be further connected to additional fluid sources such as but not limited to a cleaning solution source 572 and DI water source 574.

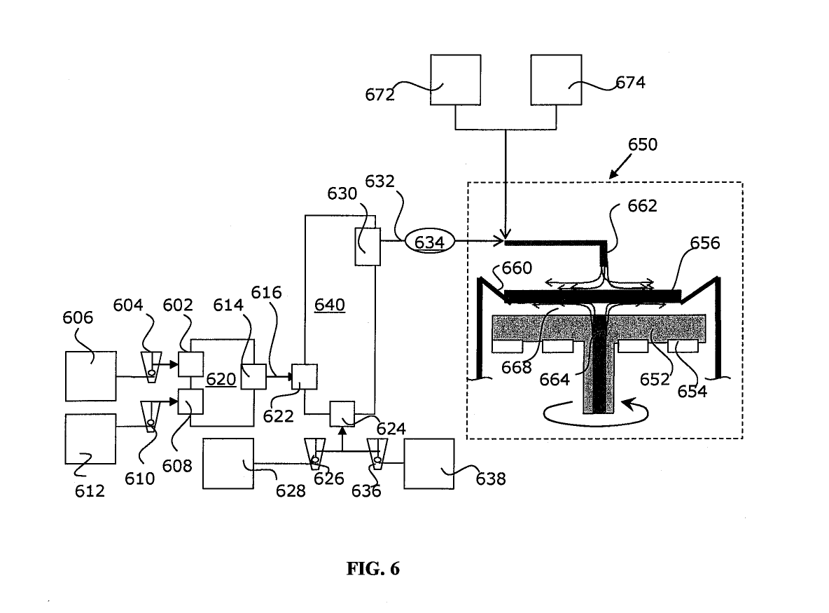

FIG. 6 is an illustration of one embodiment of an apparatus for supplying an ozonated acetic acid solution to a photomask. As shown in FIG. 6 acetic acid and Dl water can be mixed prior to ozonating the solution. Such an embodiment may be preferable when an aggressive stripping solution is required for rapidly removing a layer of photoresist several hundred nm thick. Utilizing the apparatus of FIG. 6, a maximum dissolved ozone concentration can be obtained by controlling the liquid acetic acid volume percent of the solution between 0% and 100%. However, the benefits of the apparatus of FIG. 6 in comparison to FIG. 5 are particularly realized for acetic acid volume percentages above approximately 50%. Mixer 620 includes an input port 602, connected to a flow valve 604, which is flow connected to a liquid DI water source 606. In an embodiment, liquid DI water source 606 provides pure liquid DI water. In another embodiment, liquid DI water source provides carbonated liquid DI water. Mixer 620 also includes input port 608, connected to a flow valve 610, which is flow connected to an acetic acid source 612. Mixer 620 further includes an exit port 614 connected to an exit supply line 616, for flowing a liquid acetic acid solution to ozonator 640.

Exit supply line 616 is also connected to input port 622 of ozonator 640. Ozonator 640 includes an input port 624 connected to a flow valve 626, which is flow connected to an ozone gas source 628. In an embodiment, ozonator 640 further includes a flow valve 636 connected to input port 624, and further flow connected to an additional gas source 638, such as a CO₂ gas. Ozonator 640 further includes an exit port 630 connected to an exit supply line 632, for flowing an ozonated liquid acetic acid solution. An ozone ppm monitor 634 can also be including along the supply line 632 in order to monitor ozone concentration of the solution. Mask spin chamber 650 is identical to the one described in FIG. 5.

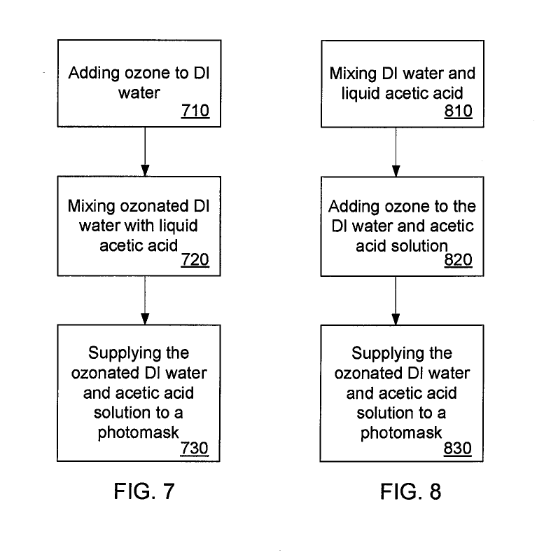

FIG. 7 illustrates an embodiment of a method for supplying an ozonated liquid acetic acid solution to a photomask utilizing the apparatus of FIG. 5. At block 710 flow valves 504 and 510 are opened to flow liquid DI water and ozone gas into a DI water compatible ozonator 520 to create an ozonated DI water solution. In an embodiment the liquid DI water flowing from source 506 and through flow valve 504 is carbonated. In an alternative embodiment, valve 536 is also opened to flow CO₂ gas into the ozonator 520. The ozonated DI water solution is then flowed through exit port 514 and into supply line 516.

At block 720 the ozonated DI water solution is mixed with liquid acetic acid. Flow valve 526 is opened to flow liquid acetic acid into mixer 540 while supply line 516 feeds into mixer 540 through input port 522. The ozonated DI water and acetic acid are then intimately mixed.

Then at block 730 the ozonated liquid acetic acid solution exits through port 530 and enters the supply line 532. The ozone concentration can be optionally monitored by monitor 534 in the supply line 532. Supply line 532 then feeds the ozonated liquid acetic acid solution to the mask spin chamber 550 and photomask 556.

It is understood that in the process of FIG. 7 and apparatus of FIG. 5 that liquid acetic acid is added after the liquid DI water has been ozonated and/or carbonated. Thus, the ozone concentration in the stripping solution is less than approximately 50 ppm since liquid DI water has a maximum solubility of ozone at approximately 50 ppm. In an embodiment, a stripping solution containing 0.1% to 50% liquid acetic acid is fed through supply line 532, and more specifically approximately 0.1% to 25%. In an embodiment, the ozonated liquid acetic acid solution comprises a liquid acetic acid volume percent of approximately 0.1% to 25%, an ozone concentration below approximately 50 ppm, a surface tension below approximately 72 dyne/cm, conductivity greater than 100 μS/cm, a flash point above approximately 100° C., and a pH less than 4.

FIG. 8 illustrates an embodiment of a method for supplying an ozonated liquid acetic acid solution to a photomask utilizing the apparatus of FIG. 6. At block 810 flow valves 604 and 610 are opened to flow liquid DI water and liquid acetic acid into mixer 620. The liquids are then intimately mixed. In an embodiment the liquid DI water flowing from source 606 and through flow valve 604 is carbonated. The liquid acetic acid solution is then flowed through exit port 614 and into supply line 616.

At block 820 the liquid acetic acid solution is then ozonated. Flow valve 626 is opened to flow gaseous ozone into the acetic acid compatible ozonator 640 through input port 624 while the acetic acid solution is fed into ozonator 640 through input port 622. In an embodiment, valve 636 is also opened to flow CO₂ gas into the ozonator 640.

Then at block 830 the ozonated liquid acetic acid solution exits through port 630 and enters the supply line 632. The ozone concentration can be optionally monitored by monitor 634 in the supply line 632. Supply line 632 then feeds the ozonated liquid acetic acid solution to the mask spin chamber 650 and photomask 656.

It is understood that in the process of FIG. 8 and apparatus of FIG. 6 that liquid acetic acid and liquid DI water can be mixed prior to ozonating. Therefore, the ozone concentration in the stripping solution is dependent upon the volume percentage of liquid acetic acid in the stripping solution. One advantage of this method is that more ozone can be dissolved in the solution. Another particular advantage of mixing liquid acetic acid with liquid DI water prior to ozonating is that the concern of ozone decomposition in the solution is reduced because acetic acid prevents ozone decomposition in the liquid solution. In an embodiment, a stripping solution comprises 100% liquid acetic acid, and therefore the addition of a scavenging agent such as CO₂ is unnecessary. In an embodiment, where a more aggressive stripping solution is desired, a stripping solution containing between approximately 50% to 100% liquid acetic acid is fed through supply line 632. More specifically, the ozonated liquid acetic acid solution comprises a liquid acetic acid volume percent above 50% and less than 70% and has an ozone concentration above 125 ppm and less than 155 ppm, a surface tension above approximately 38 dyne/cm and less than approximately 45 dyne/cm, a conductivity greater than 100 μS/cm, a flash point above approximately 60° C. to 70° C., and a pH less than 4. Such an embodiment may be preferable for rapidly removing a layer of photoresist several hundred nm thick in less than five minutes. In an embodiment, the solution removes a layer of photoresist several hundred nm thick in greater than one minute. In an embodiment the ozonated liquid acetic acid solution comprises a liquid acetic acid volume percent above 60% and less than 70%.

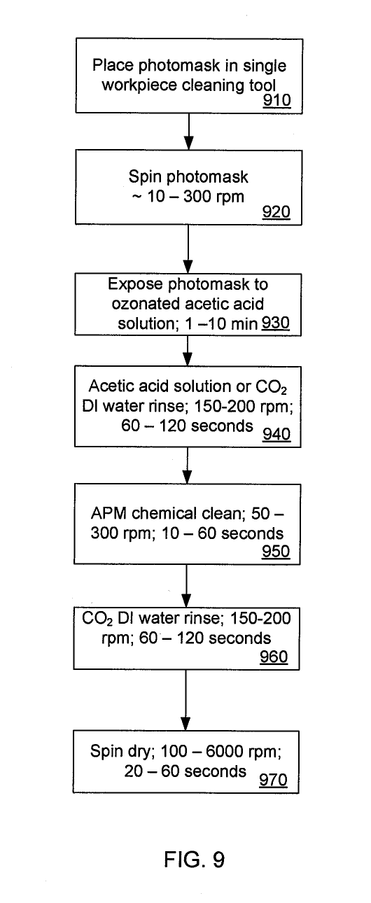

FIG. 9 illustrates an embodiment for a method of stripping a layer of photoresist from a photomask utilizing the apparatus of either FIG. 5 or FIG. 6. For simplicity only, the method of FIG. 9 is discussed using the apparatus of FIG. 5. Photomask 556 is placed on a photomask holder 560 in a single workpiece cleaning tool 550 (See FIG. 9, 910). In an embodiment, photomask 556 is placed with the side containing a photoresist layer (such as layer 110 or 112 of FIG. 1) facing up toward nozzle 562. The photomask 556 is then rotated between approximately 10 rpm to 300 rpm (see FIG. 9, 920).

Then as shown in block 930 an ozonated liquid acetic acid stripping solution is dispensed from nozzle 562 for approximately 1 to 10 minutes. In an embodiment, the ozonated liquid acetic acid solution comprises a liquid acetic acid concentration of approximately 0.1% to 50%, more specifically 0.1% to 25%. When the liquid acetic acid and DI water are mixed and then ozonated, the solution comprising 0.1% to 25% acetic acid has a concentration up to approximately 87 ppm dissolved ozone. When the DI water is ozonated prior to mixing with the liquid acetic acid, the solution comprising 0.1% to 25% acetic acid has a concentration up to approximately 50 ppm ozone.

In an embodiment where a more aggressive strip rate is desired, the ozonated acetic acid solution comprises liquid acetic between approximately 50% to 100%, more specifically greater than 50% and less than 70%, and an ozone concentration greater than 125 ppm and less than 155 ppm. The backside of the photomask can also be rinsed or stripped at this time by flowing a rinse or stripping solution into the gap 568. In an embodiment, the stripping solution flowed into gap 568 is the same solution as applied to the top surface of photomask 556 by nozzle 562. In an embodiment, a DI water rinse solution is flowed into gap 568. In another embodiment, a rinse solution comprising liquid acetic acid is flowed into gap 568. In an embodiment the rinse solution comprises liquid acetic acid and DI water. In another embodiment the rinse solution comprises liquid acetic acid, liquid water, and/or approximately 1700 ppm to 1800 ppm CO₂. In an embodiment liquid acetic acid is present at approximately 0.1% to 25% by volume. At the end of the operation the flow of acetic acid solution from nozzle 562 is stopped.

As set forth in block 940 the top surface of photomask 556 is then rinsed in order to remove all the chemicals from the surface of the photomask. In an embodiment the photomask is rotated at speed in the range of approximately 150 to 200 rpm, and the entire step lasts for approximately 60 to 120 seconds. The backside of the photomask can also be rinsed at this time by flowing a rinse solution into the gap 568.

In an embodiment, the rinse solution comprises liquid acetic acid. In an embodiment the rinse solution comprises liquid acetic acid and water. In another embodiment the rinse solution comprises liquid acetic acid, liquid water, and CO₂. For example, the rinse solution may comprise approximately 0.1% to 25% liquid acetic acid and approximately 1700 ppm to 1800 ppm CO₂. In another embodiment the rinse solution comprises carbonated DI water. For example, the rinse solution may comprise DI water and approximately 1700 ppm to 1800 ppm CO₂. The flow of rinse solution to the top surface of photomask 556 is then stopped.

Following the rinse operation the top surface of photomask 556 is exposed to a chemical clean as set forth in block 950. In an embodiment the chemical clean is an ammonia/peroxide mixture (APM) chemical clean and is applied to photomask 556 for approximately 10 to 60 seconds, while rotating the photomask at a speed in the range of approximately 50 to 300 rpm. For example, the APM chemical clean can have a composition of 1:2:50-500 (Ammonia or AM1: H₂O₂: DI water). One function of the APM clean is to remove acetic acid residues. The backside of the photomask can also be rinsed or exposed to the APM chemical clean at this time by flowing a rinse or APM solution into the gap 568.

Then the photomask 556 is rinsed again at block 960. In an embodiment the rinse solution comprises carbonated DI water. For example, the rinse solution may comprise DI water and approximately 1700 ppm to 1800 ppm CO₂. In an embodiment the rinse is applied to the photomask for approximately 60 to 120 seconds, while rotating the photomask at a speed in the range of approximately 150 to 200 rpm.

Following the cleaning operation, the flow of all solutions is stopped, and the photomask 556 is spun dry as set forth in block 970. The photomask 556 is dried by spinning at very high rotation speeds between 100 rpm to 6000 rpm, more specifically around 3000 rpm, for about 20 to 60 seconds and using air flow around the photomask to dry the photomask. If desired, N₂ and/or IPA vapor may be blown on the photomask to assist in the drying of the photomask.

It is to be appreciated that during all stripping, rinsing, and cleaning operations that megasonic transducers 554 may also be turned to produce acoustic waves while flowing solution onto the photomask. In one embodiment, megasonic energy is only applied during the cleaning operation. The transducers 554 produce acoustic waves that travel through plate 552, through the liquid filled gap 568, and through photomask 556 and into the solution on the photomask 556 top surface to enhance cleaning of the photomask 556. The megasonic waves entering the liquid filled gap 568 also help to clean the backside of the photomask 556.

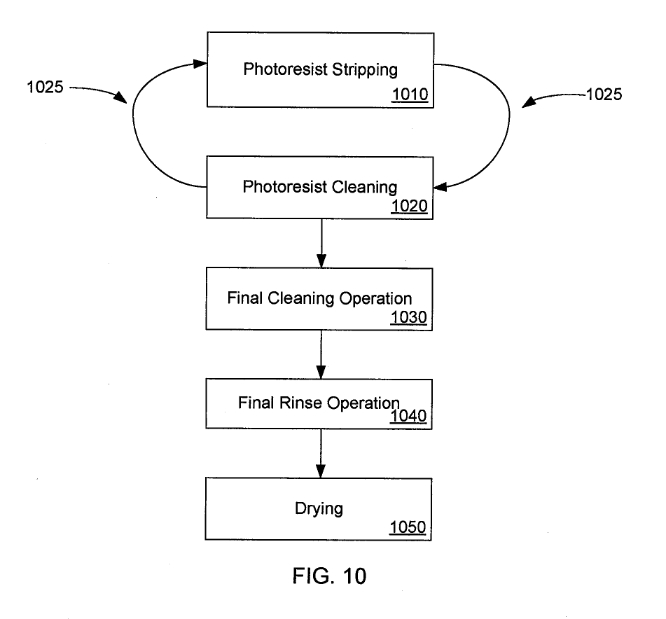

FIG. 10 illustrates another embodiment for a method of stripping a layer of photoresist from a photomask. In an embodiment, the method of FIG. 10 is particularly useful for post-etch stripping of a photoresist layer where the photoresist layer is utilized as an etching mask. As shown in FIG. 1C selective etch chemistries can be utilized to selectively etch the ARC layer 108, Cr layer 106 and phase shift layer 104. Likewise, selective etch chemistries can be utilized to selectively etch the ARC layer 108 and Cr layer 106 in FIG. 1G. In an embodiment, one or more plasma etches are used to etch layers 108, 106, and 104 of FIG. 1C utilizing photoresist layer 110 as an etching mask, which results in the formation of a hard crust (not shown) on the top surface of photoresist layer 110 which is difficult to remove. A similar etching process may be utilized to etch layers 108 and 106 of FIG. 1G utilizing photoresist layer 112 as an etching mask. In such an embodiment, a series of alternating exposures to a stripping solution and cleaning solution is performed in order to remove photoresist layer 110 as shown in FIG. 1D, and photoresist layer 112 as shown in FIG. 1H.

As shown by arrows 1025, the stripping method includes a series of alternating exposures to a stripping solution (stripping operation 1010) and a cleaning solution (cleaning operation 1020) until the photoresist is substantially or completely removed from the photomask. In an embodiment the stripping solution and cleaning solution can be applied to a photomask containing a layer of photoresist (layer 110 or 112) in a consecutive alternating manner 4 to 30 times each until the layer of photoresist is substantially removed. For both positive and negative photoresists, it is theorized that, in accordance with embodiments of the invention, the abbreviated stripping operation 1010 oxidizes and dissociates the surface of the photoresist and the abbreviated cleaning operation 1020 removes the modified surface layer resulting in significantly enhanced removal rates.

In one embodiment, the stripping solution is an ozonated liquid acetic acid solution comprising a liquid acetic acid concentration between approximately 0.1% to 50%, and more specifically 0.1% to 25% with an ozone concentration below approximately 87 ppm, and a conductivity greater than 100 μS/cm. In an embodiment, the stripping solution has an ozone concentration below approximately 50 ppm. In an embodiment, the cleaning solution comprises ammonium hydroxide, hydrogen peroxide, and water. In an embodiment, the cleaning solution may have a composition of 1:2:50-500 (Ammonia or AM1: H₂O₂: DI water). In an embodiment, the cleaning solution further includes a chelating agent and a surfactant. The ozonated liquid acetic acid solution and the cleaning solution are alternately applied until the photoresist (such as layer 110 or 112 of FIG. 1) is substantially or completely removed from the photomask.

In an embodiment, the stripping solution can change throughout the process. For example, in one embodiment, a stripping solution of ozonated liquid acetic acid solution can be applied to the photomask, followed by a cleaning solution, followed by a stripping solution of SPM, followed by a cleaning solution and thereafter repeated using different combinations of stripping solution until the photoresist is substantially or completely removed from the photomask. Similarly, in another embodiment, an ozonated liquid acetic acid stripping solution can be applied to the photomask, followed by a cleaning solution of AM-clean™, followed by an ozonated liquid acetic acid stripping solution, followed by a cleaning solution of DI water and thereafter repeated using different combinations of cleaning solution until the photoresist is substantially or completely removed from the photomask. Any combination of stripping and cleaning solutions is contemplated by embodiments of the present invention.

The ozonated liquid acetic acid solution and the cleaning solution are alternately applied until the photoresist (such as layer 110 or 112 of FIG. 1) is substantially or completely removed from the photomask. The number of applications may depend on the concentration of acetic acid and ozone in the stripping solution. In one embodiment, the stripping solution comprises greater than 50% and less than 70% acetic acid, and the stripping and cleaning solutions are applied to the photomask containing a layer of photoresist (layer 110 or 112) in a consecutive alternating manner 4 to 12 times. In one embodiment, the stripping solution comprises approximately 0.1% to 50% acetic acid, and the stripping and cleaning solutions are applied to the photomask containing a layer of photoresist (layer 110 or 112) in a consecutive alternating manner 4 to 30 times.

Following a series of consecutive alternating applications of a stripping solution and a cleaning solution, the photomask is subjected to a final cleaning operation as set forth in block 1030. The final cleaning operation can be performed with, for example, DI water or an APM cleaning solution including SC-1 or AM-clean™. In addition, the final cleaning operation can be performed using megasonic power at above 350 kHz for a period of between approximately 60 seconds and 120 seconds.

Following the final cleaning operation, the photomask is rinsed as set forth in block 1040. The rinsing operation is performed with, for example, carbonated DI water at a speed of approximately 150 to 200 rpm for a period between approximately 60 seconds and 120 seconds.

Following the rinsing operation, the photomask is subjected to a drying operation as set forth in block 1050. The drying operation can be, for example, spin drying or the like techniques. In spin drying, the photomask rotates 100 to 6000 rpm, more specifically around 3000 rpm, for about 20 to 60 seconds and using air flow around the photomask to dry the photomask. If desired, N₂ and/or IPA vapor may be blown on the photomask to assist in the drying of the photomask.

While embodiments of the invention described herein disclosed solutions comprising liquid acetic acid (CH₃COOH) and the associated benefits of the compound, it is to be appreciated that similar results could alternatively be obtained using similar organic acids such as, but not limited to, acetic anhydride or other water miscible carboxylic acids containing 1 to 4 carbon atoms. Although the present invention has been described in language specific to structural features and/or methodological acts, it is to be understood that the invention defined in the appended claims is not necessarily limited to the specific features or acts described. The specific features and acts disclosed are instead to be understood as particularly graceful implementations of the claimed invention useful for illustrating the present invention.

1. A stripping solution comprising:
liquid DI water;
less than 70 volume percent liquid acetic acid; and
ozone.

2. The stripping solution of claim 1 wherein said stripping solution comprises approximately 0.1 to 50 volume percent liquid acetic acid.

3. The stripping solution of claim 2 wherein said stripping solution comprises approximately 0.1 to 25 volume percent liquid acetic acid.

4. The stripping solution of claim 2 wherein said stripping solution has a flash point above approximately 100° C., a conductivity greater than 100□S/cm, a surface tension below 72 dyne/cm, and an ozone concentration below 50 ppm.

5. The stripping solution of claim 1 wherein said stripping solution comprises greater than 50 volume percent liquid acetic acid.

6. The stripping solution of claim 5 wherein said stripping solution has a flash point above approximately 60° C., a conductivity greater than 100□S/cm, a surface tension below 45 dyne/cm, and an ozone concentration above 125 ppm.

7. A method for stripping a layer of photoresist from a photomask comprising:
placing a photomask in a processing tool, said photomask having a photoresist layer disposed thereon; and
exposing said photomask to a stripping solution comprising:
liquid DI water;
less than 70 volume percent liquid acetic acid; and
ozone.

8. The method of claim 7 further comprising:
dissolving said ozone into said liquid water to create an ozonated liquid DI water solution; and
mixing said ozonated liquid DI water solution with said liquid acetic acid to create said stripping solution.

9. The method of claim 8 wherein said stripping solution comprises approximately 0.1 to 25 volume percent liquid acetic acid.

10. The method of claim 7 further comprising:
mixing said liquid DI water and said liquid acetic acid to create a liquid acetic acid solution comprising less than 70 volume percent liquid acetic acid; and
dissolving said ozone in said liquid acetic acid solution to create said stripping solution.

11. The method of claim 10 wherein said stripping solution comprises greater than 50 volume percent liquid acetic acid.

12. The method of claim 7 further comprising:
rinsing said photomask with a first rinse solution after exposing said photomask to said stripping solution;
exposing said photomask to a cleaning solution comprising NH₄OH, H₂O₂, and DI water;
rinsing said photomask with a second rinse solution; and
drying said photomask.

13. The method of claim 12 wherein said first rinse solution comprises liquid acetic acid and liquid DI water, and said second rinse solution comprises liquid DI water and CO₂.

14. The method of claim 13 wherein said second rinse solution comprises approximately 1700 ppm to 1800 ppm CO₂.

15-19. (canceled)

20. A method for processing a workpiece comprising:
providing a workpiece with a photoresist layer disposed thereon;
applying an ozonated liquid acetic acid solution to said workpiece during a first time interval;
applying a cleaning solution comprising ammonium hydroxide and hydrogen peroxide to said workpiece during a second time interval; and
repeating said ozonated liquid acetic acid solution and said cleaning solution applications in consecutive intervals until said photoresist layer is substantially removed from said workpiece.

21. The method of claim 20, wherein said repeating is 4 to 30 times.

22. The method of claim 20, wherein said first time interval is between 30 seconds and 120 seconds, and said second interval is between 8 seconds and 15 seconds.

23. The method of claim 20 further comprising exposing said photoresist layer to a plasma etching process prior to applying said ozonated liquid acetic acid solution to said workpiece.

24. The method of claim 20, wherein said workpiece is a photomask and said ozonated liquid acetic acid solution is diluted with at least 50 volume percent liquid H₂O.

25. The method of claim 20, wherein said ozonated liquid acetic acid solution comprises approximately 0.1 to 25 volume percent liquid acetic acid.
